(19) United States
(12) Patent Application Publication
(10) Pub. No.: US 2025/0228879 A1
(43) Pub. Date: Jul. 17, 2025

(54) METHOD AND COMPOSITION FOR CONTROLLING ACNE USING RHAMNOLIPIDS

(71) Applicant: Stepan Company, Northbrook, IL (US)

(72) Inventors: Kelly Strout, Lake Zurich, IL (US); Alexander Nikoloff, Wheeling, IL (US); Thach Bao Tran, Des Plaines, IL (US); John MacKinnon, Northbrook, IL (US); Jesse Jefferis, Lincolnshire, IL (US); Cyril Bajracharya, Arlington Heights, IL (US)

(21) Appl. No.: 19/095,141

(22) Filed: Mar. 31, 2025

Related U.S. Application Data
(63) Continuation of application No. PCT/US2023/075729, filed on Oct. 2, 2023.
(60) Provisional application No. 63/412,745, filed on Oct. 3, 2022.

(51) Int. Cl. A61K 31/7032; A61K 9/00; A61K 31/05; A61K 31/222; A61K 31/327; A61K 31/60; A61K 33/04; A61P 17/10

(52) U.S. Cl. CPC A61K 31/7032; A61K 9/0014; A61K 31/05; A61K 31/222; A61K 31/327; A61K 31/60; A61K 33/04; A61P 17/10

(57) ABSTRACT

An acne treatment composition that contains a mixture of rhamnolipid salts is disclosed. The composition has efficacy against C. acnes. Also disclosed is a method for controlling, treating, or preventing acne in a subject.

### BACKGROUND OF THE INVENTION

The present technology, in general, relates to the use of biosurfactants, such as rhamnolipids, for treating, controlling, or preventing acne. More particularly, the present technology relates to a method for treating, controlling, or preventing acne by applying a composition comprising a mixture of particular rhamnolipids to skin of a subject afflicted with or susceptible to acne. The present technology also relates to a composition comprising a mixture of rhamnolipids having efficacy against Cutibacterium acnes (formerly Proprionibacterium acnes).

The current anti-acne market consists primarily of products that fall within the FDA's OTC Monograph for topical antiacne products. There are currently only four active ingredients that are approved for this category. The two most commonly used actives are salicylic acid and benzoyl peroxide. The remaining two, resorcinol and sulfur, both present significant challenges in the area of aesthetics and formulation, which preclude including them as a consumer-level anti-acne product.

Benzoyl peroxide and salicylic acid also present certain undesirable side effects when used as topical anti-acne products at Monograph level. At usage levels, both of these ingredients may pose significant skin reactions ranging from mild irritation to severe contact dermatitis and skin pealing. As a result, the benefits must be balanced against the side effects. It would be desirable to have an active ingredient for acne treatment compositions that can provide equivalent or better activity against C. acnes without the associated side effects that can occur with benzoyl peroxide and salicylic acid.

Another common treatment option for treating or controlling acne is a prescription strength, broad-spectrum non-selective antibiotic. Apart from the disadvantage that a broad-spectrum antibiotic requires a doctor's prescription, a non-selective antibiotic reduces the total microbiome, including bacteria that may be beneficial. It would be desirable to have a non-prescription active that can target C. acnes specifically and minimize disruption of the total microbiome.

There has been a recent trend to formulate products with ingredients that are based on renewable raw materials. Such ingredients are considered “green” or “natural”, since they are derived from renewable and/or sustainable sources. As a result, they are more environmentally friendly than ingredients derived from fossil fuels or other non-renewable sources. An ingredient having a high Biorenewable Carbon Index (BCI), such as greater than 80, indicates that the ingredient contains carbons that are derived primarily from plant, animal, or marine-based sources.

Rhamnolipids are interface-active glycolipids produced by various bacterial species and are an example of a “green” ingredient, since they can be prepared by means of fermentation based on renewable raw materials. It would be desirable to provide compositions that include active ingredients derived from renewable sources, such as rhamnolipids, that can be used for controlling acne.

Applicants have determined that particular mixtures of rhamnolipid salts can meet the above objectives while also advancing UN Sustainability Goals (“SDG”). The rhamnolipid salt mixtures of the present technology contribute to better health and well-being by delivering equal or better efficacy against C. acnes than other common acne treatments, without the associated side effects, and better efficacy compared to other rhamnolipids. The rhamnolipid salt mixtures are advantageously bio-based, renewably sourced actives obtained from a bacterial fermentation process that generates biodegradable waste products that are less impactful on the environment. These benefits further SDG #3 (Good Health and Well-being) and SDG #12 (Responsible Consumption and Production).

### BRIEF SUMMARY OF THE INVENTION

In one aspect, the present technology provides an acne treatment composition comprising a mixture of purified rhamnolipids in an amount of about 0.01% to about 99% active weight, based on the total weight of the composition; and at least one dermatologically acceptable carrier, wherein the mixture of rhamnolipids comprises salts of mono-rhamnolipids and di-rhamnolipids in a weight ratio of about 10:90 to about 47:53 mono-rhamnolipids: di-rhamnolipids, an amount of Rha-C10-C10 mono-rhamnolipid salt of about 5% to about 38% by weight, based on the total weight of the rhamnolipids in the mixture of rhamnolipids, and an amount of RhaRha-C10-C10 di-rhamnolipid salt of about 34% to about 80% by weight, based on the total weight of the rhamnolipids in the mixture of rhamnolipids. In some embodiments, the mixture of rhamnolipids comprises salts of mono-rhamnolipids and di-rhamnolipids in a weight ratio of 40:60 to 45:55, alternatively 40:60 to 48:52, an amount of Rha-C10-C10 mono-rhamnolipid salt of about 29% to about 40% by weight, alternatively about 29% to 37.5% by weight, and an amount of RhaRha-C10-C10 di-rhamnolipid salts of about 34% to about 45% by weight, alternatively about 36% to about 45% by weight, based on the total weight of the rhamnolipids in the mixture of rhamnolipids.

A further aspect of the present technology provides a method for treating, controlling or preventing acne in a subject afflicted with or susceptible to acne comprising administering to the subject an effective amount of a composition comprising a mixture of purified rhamnolipid salts, thereby treating, controlling, or preventing acne in the subject, wherein the mixture of purified rhamnolipids comprises mono-rhamnolipid salts and di-rhamnolipid salts in a weight ratio of about 10:90 to about 47:53 mono-rhamnolipids: di-rhamnolipids, an amount of Rha-C10-C10 mono-rhamnolipid salt of about 5% to about 38% by weight, based on the total weight of the mixture of rhamnolipids, and an amount of RhaRha-C10-C10 di-rhamnolipid salt of about 34% to about 80% by weight, based on the total weight of the mixture of rhamnolipids. In some embodiments, the mixture of purified rhamnolipids comprises salts of mono-rhamnolipids and di-rhamnolipids in a weight ratio of 40:60 to 45:55, alternatively 40:60 to 48:52, an amount of Rha-C10-C10 mono-rhamnolipid salt of about 29% to about 40% by weight, alternatively about 29% to 37.5% by weight, and an amount of RhaRha-C10-C10 di-rhamnolipid salts of about 34% to about 45% by weight, alternatively about 36% to about 45% by weight, based on the total weight of the rhamnolipids in the mixture of rhamnolipids.

### BRIEF DESCRIPTION OF THE DRAWINGS

FIG. 1 is a graph showing the Logio reduction of C. acnes after treatment with rhamnolipid test samples and control samples.

FIG. 2 is a graph showing the enumeration of C. acnes after treatment with rhamnolipid test and comparative samples and control samples.

FIG. 3 is a graph showing the Logio reduction of C. acnes after treatment with rhamnolipid test and comparative samples and control samples.

FIG. 4 is a graph showing the enumeration of C. acnes after treatment with rhamnolipid test and comparative samples and control samples.

FIG. 5 is a graph showing the Logio reduction of C. acnes after treatment with rhamnolipid test and comparative samples and control samples.

### DETAILED DESCRIPTION OF THE INVENTION

### Definitions

“Biorenewable Carbon Index” (BCI) refers to a calculation of the percent carbon derived from a biorenewable resource and is calculated based on the number of biorenewable carbons divided by the total number of carbons in the entire molecule.

“Biorenewable” is defined herein as originating from animal, plant, or marine material.

The terms “active”, “% active”, and “% active weight” refer to the amount of the active ingredient without regard to the amount of water or other solvent that may be present with the ingredient.

“Antimicrobial” refers to an agent having effectiveness for controlling the growth of, reducing, and/or killing microbes, such as bacteria, virus, fungi, yeast, algae, cyanobacteria, etc.

As used herein, “effective amount” refers to an amount of an active ingredient or composition that, when administered to a subject, is capable of controlling, treating, or preventing acne when caused by C. acnes. The actual amount may vary depending on a number of factors, including, but not limited to, the severity of the acne, the age and health status of the subject, and the form of administration.

As defined herein, a “rhamnolipid” is a glycolipid that has a lipid portion that includes one or more, typically linear, saturated or unsaturated β-hydroxy-carboxylic acid moieties and a saccharide portion of one or more units of rhamnose.

The saccharide portion and the lipid portion are linked via a β-glycosidic bond between the 1-OH group of a rhamnose moiety of the saccharide portion and the 3-OH group of a β-hydroxy-carboxylic acid of the lipid portion. Thus, the carboxylic acid of one carboxylic acid moiety defines the end of the rhamnolipid. Where more than one rhamnose-moiety is included in a rhamnolipid, each of the rhamnose moieties not linked to the lipid portion is linked to another rhamnose moiety via a 1,4-β-glycosidic bond. In embodiments where two or more β-hydroxy-carboxylic acids are present in a rhamnolipid, the β-hydroxy-carboxylic acid moieties are selected independently from each other. β-hydroxy carboxylic acid moieties may in some embodiments be identical. In some embodiments, they are different from each other.

The present technology generally relates to an acne treatment composition that comprises a particular mixture of purified rhamnolipids in their salt form. Although rhamnolipids in their acidic, protonated form have shown antimicrobial activity against C. acnes, the bacteria known to contribute to the acneic condition, due to the acidic nature of the rhamnolipids, purified rhamnolipids in their neutralized salt form have not shown similar antimicrobial activity. However, the particular mixture of rhamnolipid salts in a pH neutral environment described herein surprisingly provides greater efficacy against C. acnes when applied to skin than other rhamnolipid mixtures known in the art. The rhamnolipids may have the following structure (I):

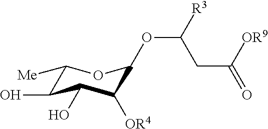

In this formula, R⁹ is a hydrogen atom (H) or an aliphatic group that has a main chain of one to about 46, such as one to about 42, one to about 40, one to about 38, one to about 36, one to about 34, one to about 30, one to about 28, including e.g. 2, 3, 4, 5, 6, 7, 8, 9, 10, 11, 12, 13, 14, 15, 16, 17, 18, 19, 20, 21, 22, 23, 24, 25, 26, 27 or 28 carbon atoms and one to about three, including two, oxygen atoms. In some embodiments, the main chain of the respective aliphatic group carries a terminal carboxylic acid group and/or an internal ester group. As an illustrative example in this regard, R⁹ may be of the formula —CH(R⁵)—CH₂—COOR⁶. In these illustrative moieties, R⁵ may be an aliphatic moiety with a main chain that has a length from 1 to about 19, such as from 1 to about 17, from 1 to about 15, from 1 to about 13, about 2 to about 13, about 3 to about 13 or about 4 to about 13, including e.g. 2, 3, 4, 5, 6, 7, 8, 9, 10, 11 or 12 carbon atoms. R⁴ in formula (I) is a hydrogen atom (H), or a rhamnopyranosyl moiety. R⁶ is a hydrogen atom.

The term “aliphatic” means, unless otherwise stated, a straight or branched hydrocarbon chain, which may be saturated or mono-or poly-unsaturated and include heteroatoms. The term “heteroatom” as used herein means an atom of any element other than carbon or hydrogen. Herein, an unsaturated aliphatic group contains one or more double bonds (alkenyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1to about 5, to about 10, to about 15 or to about 20 carbon atoms. Examples of alkenyl moieties are straight-chain or branched hydrocarbon moieties that contain one or more double bonds. Alkenyl moieties generally contain about two to about twenty carbon atoms and one or more, for instance two, double bonds, such as about two to about ten carbon atoms, and one double bond. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3-dimethylbutyl. Both the main chain as well as the branches may furthermore contain heteroatoms as for instance N, O, S, Se or Si, or a carbon atom may be replaced by one of these heteroatoms. An aliphatic moiety may be substituted or unsubstituted with one or more functional groups. Substituents may be any functional group, as for example, but not limited to, amino, amido, carbonyl, carboxyl, hydroxyl, nitro, thio and sulfonyl.

In a more particular embodiment, the rhamnolipid salts in said structure have the structure (II):

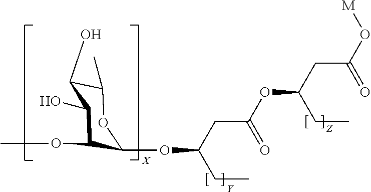

wherein x is 1 or 2, y is 4, 6 or 8, z is 4, 6, or 8, and M is H, or a metal, such as alkali metals Li, Na, or K, alkali earth metals Mg or Ca, or transition metals Mn, Fe, Cu, or Zn. In the cases of the alkali earth and transition metals, multiple rhamnolipid salt moieties may associate with each metal.

The mixture of rhamnolipids comprises a mixture of mono-rhamnolipids and di-rhamnolipids. The mono-rhamnolipids may be present in an amount of about 10% to about 48%, alternatively about 15% to about 48%, alternatively about 20% to about 48%, alternatively about 30% to about 48%, alternatively about 40% to about 48%, alternatively about 43% to about 48% alternatively about 10% to about 47%, alternatively about 15% to about 47%, alternatively about 20% to about 47%, alternatively about 30% to about 47%, alternatively about 40% to about 47%, alternatively about 43% to about 47% alternatively about 15% to about 45%, alternatively about 20% to about 45%, alternatively about 30% to about 45%, alternatively about 40% to about 45%, alternatively about 43% to about 45% based on the total weight of rhamnolipids in the mixture.

The di-rhamnolipids may be present in an amount of about 52% to about 90% by weight, alternatively about 52% to about 85%, alternatively about 52% to about 80%, alternatively about 52% to about 70%, alternatively about 52% to about 60%, alternatively about 52% to about 57%, alternatively about 53% to about 90% by weight, alternatively about 53% to about 85%, alternatively about 53% to about 80%, alternatively about 53% to about 70%, alternatively about 53% to about 60%, alternatively about 53% to about 57% alternatively about 54% to about 85%, alternatively about 55% to about 80%, alternatively about 55% to about 70%, alternatively about 55% to about 60%, alternatively about 55% to about 57% by weight, based on the total weight of rhamnolipids. The amounts of mono-rhamnolipids and di-rhamnolipids in the mixture of rhamnolipids described herein are determined by HPLC. The ratio of mono-rhamnolipids: di-rhamnolipids can be from about 10:90 to about 48:52, alternatively about 47:53, alternatively about 40:60 to about 45:55. In some embodiments, the ratio of mono-rhamnolipids: di-rhamnolipids can range from 43:57 to 45:55 or 43:57 to 48:52.

The mixture of rhamnolipids preferably comprises mono (where x=1) and di (where x=2) rhamnolipids where y and z are 6 and M is H or Na. The mono-rhamnolipid may be referred to as Rha-C10-C10, with a formula of C₂₆H₄₈O₉. The IUPAC Name is 3-[3-[(2R,3R,4R,5R,6S)-3,4,5-trihydroxy-6-methyloxan-2-yl]oxydecanoyloxy]decanoic acid. The di-rhamnolipid may be referred to as RhaRha-C10-C10, with a formula of C₃₂H₅₈O₁₃. The IUPAC name is 3-[3-[4,5-dihydroxy-6-methyl-3-(3,4,5-trihydroxy-6-methyloxan-2-yl)oxyoxan-2-yl]oxydecanoyloxy]decanoic acid. Rha-C10-C10 may be present in the mixture in an amount of about 5% to 38%, alternatively about 10% to about 38%, alternatively about 15% to about 37.5%, alternatively about 20% to about 37.5%, alternatively about 25% to about 37.5%, alternatively about 29% to about 37.5%, alternatively about 35% to about 37% by weight based on the total weight of rhamnolipids. RhaRha-C10-C10 may be present in the mixture in an amount of about 34% to about 80%, alternatively about 35% to about 75%, alternatively about 35% to about 65%, alternatively about 35% to about 60%, alternatively about 35% to about 55%, alternatively about 35.5% to about 50%, alternatively about 36% to about 45%, alternatively about 36% to about 38% by weight based on the total weight of rhamnolipids.

In addition to Rha-C10-C10 and RhaRha-C10-C10, the mixture of rhamnolipids may comprise RhaRha-C10-C12 in an amount of about 5% to about 15%, alternatively about 9% to about 12%, alternatively about 10% to about 12.5% by weight based on the total weight of rhamnolipids, and Rha-C10-C12 in an amount of about 0.2% to about 6% by weight, alternatively about 2% to about 5%, alternatively about 3.5% to about 5% by weight based on the total weight of rhamnolipids. The mixture of rhamnolipids may also comprise RhaRha-C10-C12: 1 in an amount of about 0.2% to about 5% by weight, alternatively 1% to about 4% by weight, based on the total weight of rhamnolipids, an amount of RhaRha-C8-C10 in the range of about 0.2% to about 5% by weight, alternatively about 1% to about 4% by weight, based on the total weight of rhamnolipids, and an amount of Rha-C8-C10 in the range of about 0.2% to about 5% by weight, alternatively about 1% to about 4% by weight, based on the total weight of rhamnolipids.

In a still further aspect, the rhamnolipid compositions disclosed herein display a characteristic Minimum Inhibitory Concentration (MIC) for C. acnes, determined as set forth in Example 3 (MIC/MBC Testing). In this aspect, the rhamnolipid compositions disclosed herein display a MIC for C. acnes, determined in accordance with the methodology set forth in Example 3 (MIC/MBC Testing), of from about 21 ppm to about 98 ppm, from about 21 ppm to about 90ppm, from about 21 ppm to about 80 ppm, from about 21 ppm to about 70 ppm, from about 21ppm to about 50 ppm, from about 40 ppm to about 98 ppm, from about 40 ppm to about 90 ppm, from about 40 ppm to about 80 ppm, from about 40 ppm to about 70 ppm, or from about 40 ppm to about 50 ppm.

In another aspect, the rhamnolipid compositions disclosed herein display a characteristic Minimum Bacteriocidal Concentration (MBC) for C. acnes, determined as set forth in Example 3 (MIC/MBC Testing). In this aspect, the rhamnolipid compositions disclosed herein display a MBC for C. acnes, determined in accordance with the methodology set forth in Example 3 (MIC/MBC Testing), of from about 61 ppm to about 296 ppm, from about 61 ppm to about 200ppm, from about 61 ppm to about 150 ppm, from about 61 ppm to about 125 ppm, from about 75ppm to about 296 ppm, from about 75 ppm to about 200 ppm, from about 75 ppm to about 150ppm, from about 75 ppm to about 125 ppm, from about 100 ppm to about 296 ppm, from about 100 ppm to about 200 ppm, from about 100 ppm to about 150 ppm, from about 100 ppm to about 125 ppm, from about 120 ppm to about 296 ppm, from about 120 ppm to about 200 ppm, from about 120 ppm to about 150 ppm, or from about 120 ppm to about 125 ppm.

In another aspect, the rhamnolipid compositions disclosed herein display a characteristic percent reduction in C. acnes Colony Forming Units (CFU) per mL, using significantly reduced amounts of rhamnolipid and contact times, as set forth in Example 7 (Time-Dependency Comparative Testing). In this aspect, the rhamnolipid compositions disclosed herein display a percent reduction in C. acnes Colony Forming Units (CFU) per mL of from 70% to about 99.999%, alternatively from 80% to about 99.999%, alternatively from 85% to about 99.999%, alternatively from 90% to about 99.999%, alternatively from 95% to about 99.999%, or alternatively from 99% to about 99.999%, as determined in accordance with the methodology set forth in Example 7 (using a reduced rhamnolipid amount of 0.003% by weight (based on the total weight of the composition), and a reduced contact time of 15 minutes). Also, with respect to this aspect, the rhamnolipid compositions disclosed herein display a reduction in C. acnes Colony Forming Units (CFU) per mL of from about 0.52 to about 5.0 log₁₀ reduction, alternatively from about 0.70 to about 5 log₁₀ reduction, alternatively from about 0.82 to about 5 log₁₀ reduction, alternatively from about 1.0 to about 5 log₁₀ reduction, alternatively from about 1.3 to about 5 log₁₀ reduction, alternatively from about 2.0 to about 5 log₁₀ reduction, as determined in accordance with the methodology set forth in Example 7 (using a reduced rhamnolipid amount of 0.003% by weight (based on the total weight of the composition), and a reduced contact time of 15 minutes).

The rhamnolipids may be produced from a rhamnolipid-producing microorganism that has the capacity to synthesize/produce rhamnolipids under suitable conditions. Such microorganisms include, but are not limited to, bacteria, particularly bacteria of the phyla Pseudomonadota, Actinobacteria, Fimicutes, and Proteobacteria. The rhamnolipids are naturally derived and therefore have a BCI of 100. In a particular embodiment, the rhamnolipid-producing microorganism for producing the rhamnolipids is Pseudomonas aeruginosa. Methods of culturing the rhamnolipid-producing bacteria and the production of rhamnolipids from fermentation are known in the art from, for example U.S. Pat. Nos. 11,142,782 and 10,144,943,incorporated herein by reference in their entirety. Methods of purifying the rhamnolipids are also known in the art from, for example, U.S. Pat. Nos. 9,884,883 and 10,829,507,incorporated herein by reference in their entirety. The rhamnolipids in the mixture of rhamnolipids used in the present technology are separated from the fermentation medium, washed, deodorized, decolorized, and neutralized to form the purified rhamnolipid salts.

The mixture of rhamnolipid salts can be used alone, as the sole active ingredient in the acne treatment composition. When used alone, the mixture of rhamnolipids may be in the range of about 0.003% to about 99% by active weight, based on the total weight of the composition, alternatively about 0.003% to about 25%, alternatively about 0.003% to about 10%, alternatively about 0.003% to about 6%, alternatively about 0.003% to about 0.1% by active weight, alternatively about 0.01% to about 99% by active weight, alternatively about 0.02% to about 25%, alternatively about 0.1% to about 10%, alternatively about 0.2% to about 6% by active weight, based on the total weight of the composition. The mixture of rhamnolipid salts may also be used as a co-active in combination with another anti-acne active ingredient, such as salicylic acid, benzoyl peroxide, resorcinol, resorcinol monacetate, sulfur, or combinations thereof. When used with another co-active, the mixture of rhamnolipids may be in the range of about 0.003% to about 95% by active weight, based on the total weight of the composition, alternatively about 0.003% to about 25%, alternatively about 0.003% to about 10%, alternatively about 0.003% to about 6%, alternatively about 0.003% to about 0.1%, alternatively about 0.01% to about 95% alternatively about 0.02% to about 25%, alternatively about 0.1% to about 10%, alternatively about 0.2% to about 6% by active weight, based on the total weight of the composition. The combination of the mixture of rhamnolipid salts and another co-active ingredient may help to alleviate the irritation potential of the co-active without reducing or inhibiting its activity. Combining the mixture of rhamnolipid salts with another co-active ingredient may also allow for the reduction of the other co-active ingredient, which can also help to reduce the overall irritation potential.

The acne treatment compositions can be formulated into any treatment form commonly used for dermatological applications. For example, the compositions can be in the form of an aqueous solution, suspension, cream, lotion, gel, paste, spray, cream, foam or emollient, or impregnated onto a pad or wipe.

The acne treatment compositions of the present technology also include at least one carrier suitable for personal or dermatological care to bring the total percentage of the composition to 100%. As will be appreciated by at least those skilled in the art, a variety of carriers, vehicles, diluents, and the like are suitable for use in the practice of the present technology. Thus, it will also be appreciated that the terms “carrier”, “vehicle”, and “diluent” are to be considered non-exhaustive and interchangeable with respect to the present technology and in describing the various formulations, applications, uses, and compositions thereof.

Water is a suitable carrier, and can be deionized water, hard water, soft water, distilled water, tap water or combinations thereof. Water can be used alone as the carrier, or in combination with other carriers suitable for personal care, such as for example, alcohols such as ethanol, isopropanol, or benzyl alcohol; glycols such as propylene glycol, or polyethylene glycol. Other carriers can include, but are not limited to solvents, emulsifiers, or solubilizers.

When the treatment form is a cream, gel, or paste, the acne treatment compositions can include, but are not limited to, vegetable gums, starches, celluloses, waxes, silicone, silica, or clays, as carrier ingredients. When the treatment form is a spray, the composition may include a propellant.

In addition to the rhamnolipid active and carrier, the acne treatment compositions of the present technology can include optional ingredients as known in the art. Such other components or additives can include, but are not limited to, surfactants, pH adjustment agents, skin conditioners, antioxidants, preservatives, fragrances, pigments, dyes, and other excipients.

The acne treatment compositions of the present technology can have pH values in the range of about 4.0 to about 8.5, alternatively, about 5.0 to about 8.0, ideally 5.5 to 7.0.

The acne treatment compositions of the present technology may be used to treat, control, or prevent acne by applying the composition to the skin of a subject in an amount effective to treat, control, or prevent acne. “Applying” can refer to any commonly used method of application, such as, but not limited to, spreading a cream or gel containing the acne treatment composition on the surface of the skin and allowing the cream or gel to remain on the skin; spraying a liquid containing the acne treatment composition on the surface of the skin and wiping the skin or allowing the spray to remain on the skin; wiping the skin with a wipe impregnated with the acne treatment composition and allowing the composition to remain on the skin; applying a pad impregnated with the acne treatment composition and allowing the pad to remain on the surface of the skin; or an aqueous or nonaqueous liquid wash intended to treat the surface of the skin.

Dosage forms and treatment regimens using the acne treatment compositions of the present technology can vary with the type and intensity of the disease. For example, the type of acne may include non-inflammatory (e.g., comedonal) or inflammatory acne (e.g., papulopustular or nodular). The severity of the acne may also be classified as: mild (e.g., fewer than 20comedones, or fewer than 15 inflammatory lesions, or total lesion count fewer than 30); moderate (e.g., 20-100 comedones, or 15-50 inflammatory lesions, or total lesion count of between 30-125); or severe (e.g., greater than 5 nodules, or a total inflammatory count greater than 50, or a total lesion count greater than 125). In one or more embodiments, methods of treatment in accordance to the present technology may use a one, two, three, four, or more daily dosage regime. The daily dosage regimen can continue for one, two, three, four, five, six, or more weeks according to the condition and response of the patient.

The acne treatment composition of the present technology, comprising the particular mixture of rhamnolipid salts and a carrier, has efficacy against C. acnes. In some embodiments, the composition shows efficacy against C. acnes >4.6 Log₁₀ reduction (>99.997% reduction) after 1 hour and >2.95 Log₁₀ reduction (>99.89% reduction) after 15 minutes as determined in accordance with industry standards American Society for Testing and Materials (ASTM) E1054. In some embodiments, the acne treatment composition provides a minimum inhibitory concentration (MIC) of 41 ppm or less and a minimum bactericidal concentration (MBC) of 123 ppm or less against C. acnes, as determined in accordance with industry standards (Clinical and Laboratory Standards Institute (CLSI) M7, M11, and M26). The mixture of rhamnolipids in salt form also provides additional benefits. For example, since the rhamnolipid salts are at a neutral pH, they do not contribute to side effects, such as redness, peeling, dryness or other potential effects that more acidic anti-acne actives, such as salicylic acid and benzoyl peroxide, may cause. In addition, the mixture of rhamnolipids has the potential to provide overall skin wellness ranging from improved barrier function and moisture content to a rebalancing of natural resident flora and fauna that can help impair the growth of C. acnes thereby reducing the acneic condition.

One skilled in the art will recognize that modifications may be made in the present technology without deviating from the spirit or scope of the invention. The invention is further illustrated by the following examples, which are not to be construed as limiting the invention in spirit or scope to the specific procedures or compositions described therein.

### EXAMPLES

### Example 1: Efficacy Against C. Acnes

A study was conducted to determine the antimicrobial activity of a rhamnolipid mixture of the present technology alone against C. acnes, available from American Type Culture Collection (ATCC), Manassas, VA as ATCC 6919. Test samples were prepared by mixing 6.0% by weight of the rhamnolipid mixture into an aqueous base. Two samples were prepared, one sample using a rhamnolipid mixture processed by a non-solvent purification process, and the other sample using a rhamnolipid mixture processed by a solvent extraction process. The rhamnolipid mixtures in each sample comprise a weight ratio of mono-rhamnolipids to di-rhamnolipids in the range of 43:57 to 45:55. Efficacy of the test samples were compared against salicylic acid in deionized water at a concentration of 0.3%, using deionized water and a buffer as controls. The results are shown in Table 1.

TABLE 1

| | | | 1 h Exposure | | | 24 h Exposure | | |
|---|---|---|---|---|---|---|---|---|
| | | | Recovery | Reduction | | Recovery | Reduction | |
| | Tested | | Log₁₀ | Log₁₀ | | Log₁₀ | Log₁₀ | |
| | Conc. | pH | CFU/mL | CFU/mL | % | CFU/mL | CFU/mL | % |
| Count | N/A | 6.6 | 5.6 ± 0.1 | N/A | N/A | 5.3 ± 0.1 | N/A | N/A |
| Control in DI | | | | | | | | |
| Water | | | | | | | | |
| Count | N/A | 7.2 | N/A | N/A | N/A | 5.6 ± 0.2 | N/A | N/A |
| Control in | | | | | | | | |
| Buffer | | | | | | | | |
| Rhamnolipid | 6.0% | 7.2 | <10 | >4.6 | >99.997% | <10 | >4.3 | >99.995% |
| (non-solvent) | | | | | | | | |
| Rhamnolipid | 6.0% | 7.5 | <10 | >4.6 | >99.997% | <10 | >4.3 | >99.995% |
| Mixture | | | | | | | | |
| (solvent) | | | | | | | | |
| Salicylic acid | 0.3% * | 2.7 | <10 | >4.6 | >99.997% | <10 | >4.3 | >99.995% |
| in DI Water | | | | | | | | |

* solubility limit in water at 25° C.

The results in Table 1 show the rhamnolipid mixtures demonstrate effective killing of C. acnesand show efficacy comparable to salicylic acid.

### Example 2: Skin Analog Efficacy Against C. Acnes

A study was conducted to determine antimicrobial activity of the rhamnolipid mixture of the present technology against C. acnes using a skin analog as the growth substrate for C. acnes. The samples tested are shown in Table 2:

TABLE 2

| Treatment Group | Test Composition |
|---|---|
| Untreated | N/A |
| dPBS (vehicle) | Dulbecco's Phosphate Buffered |
| | Saline w/o Calcium and Magnesium |
| Acnecide gel (control) | 5% w/v Acnecide Gel |
| 2% Salicylic Acid Serum | 2% Salicylic Acid Serum |
| (control) | |
| 0.2% Rhamnolipid Mixture | 0.2 w/v Rhamnolipid Mixture |
| | in dPBS |
| 0.02% w/v Rhamnolipid | 0.02% w/v Rhamnolipid Mixture |
| Mixture | in dPBS |

The rhamnolipid mixture was processed by a non-solvent purification process, and had a mono-rhamnolipid to di-rhamnolipid weight ratio in the range of 43:57 to 45:55. The skin analog substrate was prepared by embedding primary adult human dermal fibroblasts into a fibrin matrix to produce dermal equivalents (DEs). The DEs were cultured to allow the fibroblasts to remodel the matrix. Primary neonatal human keratinocytes were applied to the DE surface and cultured under liquid for 48 hours. Labskin was cultured at the air liquid interface until a stratified epidermis was formed.

The C. acnes microorganism (NCTC 737) was incubated on reinforced clostridial agar medium with furazolidone for 4 days at 37° C. anaerobic. The Labskin was inoculated with an initial inoculm containing about 1.1×10⁸ CFU mL⁻¹ of the C. acnes bacterium in inoculation buffer (GS25). 10 microliters of the inoculum was used to colonize each Labskin unit, and the Labskin was incubated at 37° C. for 24 hours. After 24 hours, five Labskin units were left untreated, and the remainder of Labskin units were treated with 11 microliters of control or test item. All Labskin units were then incubated at 37° C. for 24 hours. Biopsy samples of 8 mm in diameter were aseptically removed from the center of each Labskin unit and viable microbial numbers were assayed by recovery appropriate culture medium.

Technical replicate counts obtained from selective solid media were averaged and used to calculate the colony forming units per square cm (CFU cm⁻²). Log₁₀ was calculated by working out the Log₁₀ value of each CFU cm⁻² value and averaging the results. Log₁₀ difference was calculated using the following formula:

Log₁₀ difference=Log₁₀(Treatment)−Log₁₀(Untreated)

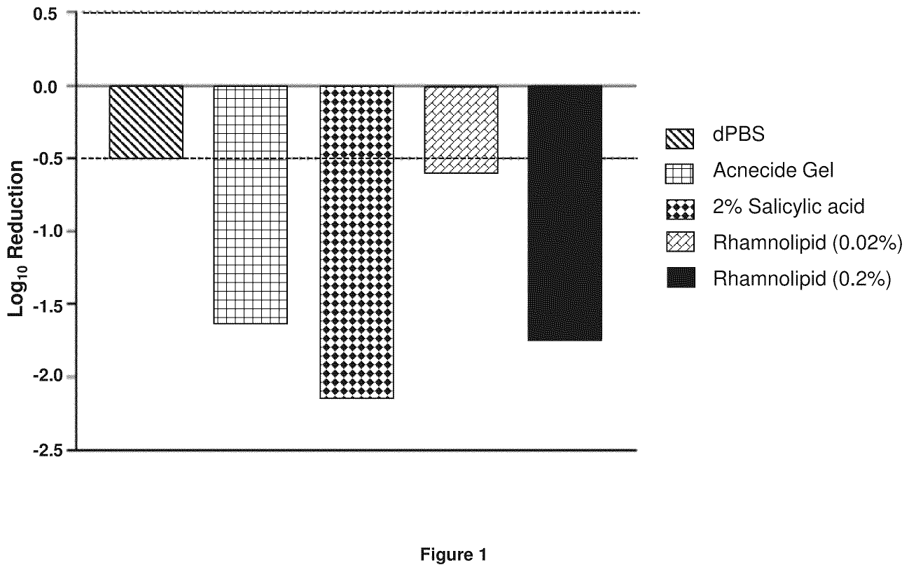

The results are shown graphically in FIG. 1. The results in FIG. 1 show that the rhamnolipid test samples have significantly reduced the population of C. acnes at both tested concentrations, although the higher concentration (0.2%) rhamnolipid sample had a greater effect on the C. acnes population. The 0.2% rhamnolipid sample was also slightly more effective than the acnecide gel control, and nearly as effective as the salicylic acid control, even though the 0.2% concentration of rhamnolipid was 10 times less than that of the acnecide gel and salicylic acid.

### Example 3: MIC/MBC Testing

A study was conducted to assess the minimum inhibitory concentration (MIC) and minimum bactericidal concentration (MBC) of the rhamnolipid mixture of the present technology (Example 3 Composition), and a commercially available rhamnolipid product comprising a mixture of rhamnolipids having a mono-rhamnolipid: di-rhamnolipid ratio of about 4:96 (Comparative Composition), against C. acnes, ATCC 6919. The rhamnolipid mixture in the Example 3 Composition was processed using a non-solvent purification process, and had a mono-rhamnolipid to di-rhamnolipid weight ratio in the range of 43:57 to 45:55. The Example 3 Composition and Comparative Composition are rhamnolipid active-only compositions diluted in deionized water.

As used herein, the MIC is the concentration of active at which no growth of the target bacteria is detected after sufficient incubation in nutrient broth media against controls. The MIC was determined in accordance with industry standards (Clinical and Laboratory Standards Institute (CLSI) M7, M11, and M26) in that samples were serially diluted, typically 1:2 or 1:3, in nutritive medium suitable for the growth of the tested microorganism and sufficiently pre-reduced to remove oxygen prior to conducting anaerobic testing. Pre-reduction was conducted by using an indirect atmospheric sequestering of oxygen using hydrogen reaction over palladium catalyst. Subsequently, samples were exposed to a minimum of 1.0E+06 CFU/mL final concentration of the test challenge microorganism. Following an initial incubation period sufficient for positive controls (test vessels with nutrient media and microorganisms; without active ingredients) to develop measurable growth, samples were evaluated for presence/absence of growth visually by turbidity assessments. Sample concentrations that did not turn turbid as compared to positive controls and were comparable to negative controls marked the concentration of inhibitory action.

As used herein, the MBC is the lowest concentration of the antibacterial composition required to kill a specific target bacterium. The MBC was determined in accordance with industry standards CLSI M7, M11, and M26. Test samples showing inhibitory effectiveness in the MIC test were further tested for bactericidal properties by neutralizing an aliquot extracted from each concentration tested and plated onto appropriate agar medium to determine presence of survivors. Samples which showed ≥3.0 Logio reduction (≥99.9% reduction) against controls after 48 hours were determined to be bactericidal concentrations. The results are shown in Table 3.

TABLE 3

| | | Results (ppm) | |
|---|---|---|---|
| Sample | Amount Tested (ppm) | MIC | MBC |
| Example 3 | 4.6-30,000 | 41 | 123 |
| Composition | | | |
| Comparative | 4.6-30,000 | 123 | 370 |
| Composition | | | |

Minimum Inhibitory Concentration (MIC) = No Growth Detected
After ≥96 h
Minimum Bactericidal Concentration (MBC) = ≥99.9% Reduction
After ≥48 h
Modified Reinforced Clostridial Broth (RCMB) pH 6.6-7.0

The results in Table 3 show that the mixture of rhamnolipids of the present technology is more effective against C. acnes than the Comparative Composition. The Example 3 Composition demonstrated an MIC of 41 ppm, which is lower than the 123 ppm of the Comparative Composition. The Example 3 Composition also demonstrated a lower MBC at 123ppm, compared to that of the Comparative Composition at 370 ppm.

### Example 4: Comparative Efficacy Testing Against C. Acnes Using Skin Analog

A study was conducted to compare the efficacy of the rhamnolipid mixture of the present technology and comparative treatments against C. acnes using a skin analog as the growth substrate for C. acnes. The samples tested are shown in Table 4:

TABLE 4

| Treatment Group | Test Composition |
|---|---|
| Untreated | N/A |
| dPBS (vehicle) | Dulbecco's Phosphate Buffered Saline w/o |
| | Calcium and Magnesium |
| Acnecide gel (control) | 5% w/v Acnecide Gel |
| 2% salicylic acid serum (control)* | 2% Salicylic Acid Serum |
| Competitive rhamnolipid mixture (49.2% active) | 0.2 w/v Competitive rhamnolipid mixture in |
| | dPBS |
| 0.2% Example rhamnolipid mixture purified by a | 0.2 w/v Example rhamnolipid mixture (non- |
| non-solvent purification process (31.1% active) | solvent process) in dPBS |
| 0.2% Example rhamnolipid mixture purified by a | 0.2 w/v Example rhamnolipid mixture (solvent |
| solvent extraction process (25% active) | process) in dPBS |
| 0.5% Example rhamnolipid mixture purified by a | 0.5 w/v Example rhamnolipid mixture (solvent |
| solvent extraction process (25% active) | process) in dPBS |
| 0.8% Example rhamnolipid mixture purified by a | 0.8 w/v Example rhamnolipid mixture (solvent |
| solvent extraction process (25% active) | process) in dPBS |

*Revolution Skincare 2% Salicylic Acid BHA Anti-Blemish Serum

The Example rhamnolipid mixtures each had a mono-rhamnolipid to di-rhamnolipid weight ratio in the range of 43:57 to 45:55. The Competitive rhamnolipid mixture had a mono-rhamnolipid to di-rhamnolipid weight ratio of 4:96.

The skin analog substrate (“Labskin”) was prepared by embedding primary adult human dermal fibroblasts into a fibrin matrix to produce dermal equivalents (DEs). The DEs were cultured to allow the fibroblasts to remodel the matrix. Primary neonatal human keratinocytes were applied to the DE surface and cultured under liquid for 48 hours. Labskin was cultured at the air liquid interface until a stratified epidermis was formed.

The C. acnes microorganism (NCTC 737) was incubated anaerobically on reinforced clostridial agar medium with furazolidone for 4 days at 37° C. The Labskin was inoculated with an initial inoculm containing about 1.1×10⁸ CFU mL⁻¹ of the C. acnes bacterium in inoculation buffer (GS25). 10 microliters of the inoculum were used to colonize each Labskin unit, and the Labskin was incubated at 37° C. for 24 hours. After 24 hours, five Labskin units were left untreated, and the remainder of Labskin units were treated with 11 microliters of control or test item. All Labskin units were then incubated at 37° C. for 24 hours. Biopsy samples of 8 mm in diameter were aseptically removed from the center of each Labskin unit and viable microbial numbers were assayed by recovery on appropriate culture medium.

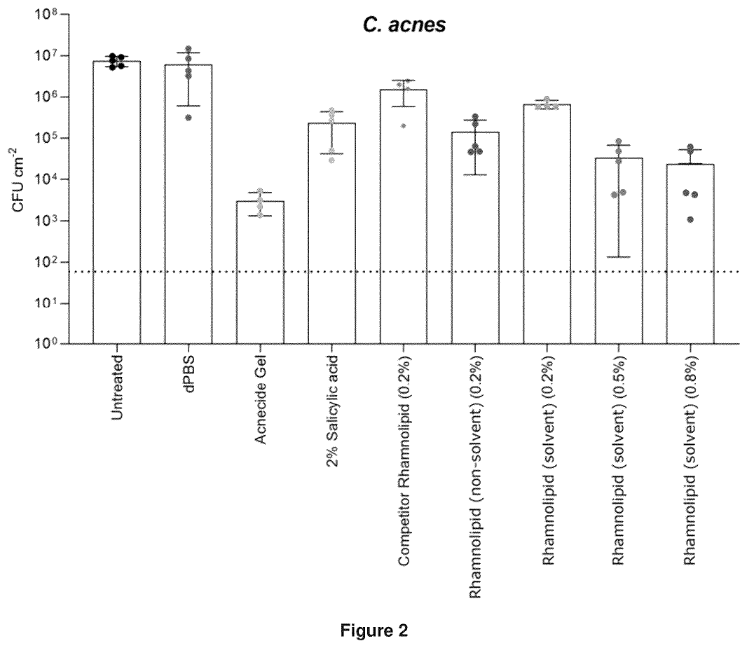

Technical replicate counts obtained from selective solid media were averaged and used to calculate the colony forming units per square cm (CFU cm⁻²). The enumeration of C. acnes for the control and test samples is shown graphically in FIG. 2. The graphed results show that, when compared with “Untreated”, there is minimal reduction in the average amount of C. acnes caused by the dPBS sample, and a significant reduction in the average amount of C. acnes for the positive control (acnecide gel) and each of the test samples.

Logio was calculated by working out the Logio value of each CFU cm⁻² value and averaging the results. Log₁₀ difference was calculated using the following formula:

Log₁₀ difference=Log₁₀(Treatment)−Log₁₀(Untreated)

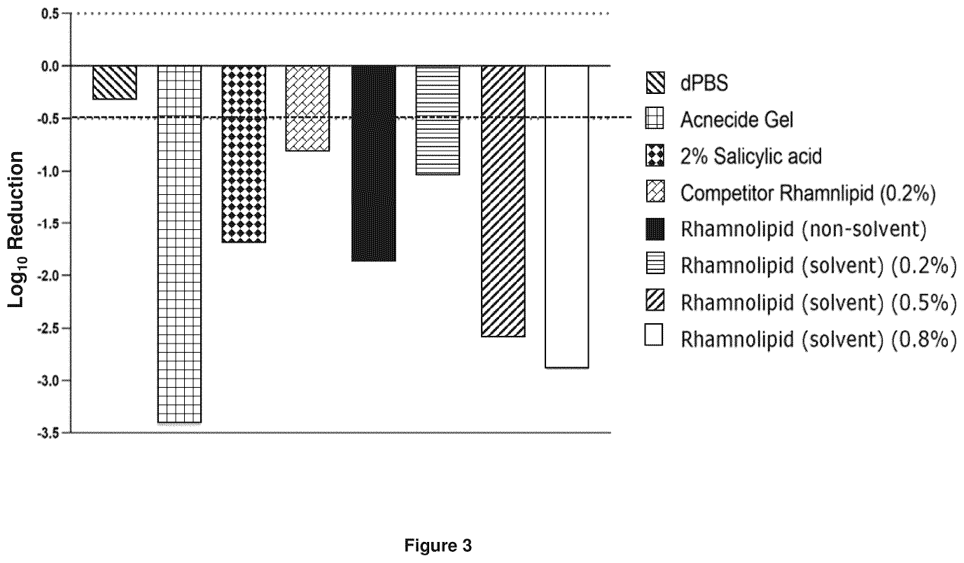

The Log₁₀ difference results are shown graphically in FIG. 3. The Log₁₀difference analysis enables a holistic comparison between the treatment samples. From this analysis, it is possible to establish a biologically significant change caused by the treatment samples in relation to the “Untreated” control. A significant difference is defined as ±0.5 Log₁₀CFU cm⁻². The graphed results in FIG. 3 show that the dPBS control did not significantly reduce the population of C. acnes, whereas the acnecide gel, salicylic acid, and each of the rhamnolipid treatment samples significantly reduced the population of C. acnes. The results in FIG. 3 also show that the Example rhamnolipids of the present technology were all more effective (greater reduction in C. acnes population) than the Competitive rhamnolipid mixture, and that the 0.5% and 0.8% concentrations of the Example rhamnolipids had a greater reduction in C. acnes than any treatment other than the Acnecide gel. From FIG. 3 it is also possible to observe a dose response effect with the Example solvent-puritifed rhamnolipids, with higher concentrations having a greater effect on the C. acnes population.

### Example 5: Efficacy of Rhamnolipids Compared to Salicylic Acid and Benzoyl Peroxide

A study was conducted to compare the efficacy of the rhamnolipid mixture of the present technology with salicylic acid and benzoyl peroxide, two common over-the-counter anti-acne actives. The samples tested are shown in Table 5. The samples containing salicylic acid and benzoyl peroxide were additionally mixed using a tip sonicator to ensure complete dissolving or adequeate distribution of the active.

TABLE 5

| Treatment Group | Test Composition |
|---|---|
| dPBS | Dulbecco's Phosphate Buffered Saline |
| Tween 80 (vehicle) | 20% (w/w) Tween 80 in dPBS |
| 0.5% Example rhamnolipid mixture purified by a | 0.5% w/w Example rhamnolipid mixture |
| solvent extraction process (25% active) | (solvent process) in dPBS |
| 2% salicylic acid | 2% w/w salicylic acid in 20% w/w Tween in |
| | dPBS |
| 0.5% salicylic acid | 0.5% w/w salicylic acid in 20% w/w Tween in |
| | dPBS |
| 2% salicylic acid and 0.5% Example rhamnolipid | 2% w/w salicylic acid and 0.5% w/w Example |
| mixture purified by a solvent extraction process | rhamnolipid mixture in 20% w/w Tween in |
| (25% active) | dPBS |
| 5% benzoyl peroxide | 5% w/w benzoyl peroxide in 20% w/w Tween |
| | in dPBS |
| 2.5% benzoyl peroxide | 2.5% w/w benzoyl peroxide in 20% w/w |
| | Tween in dPBS |
| 5% benzoyl peroxide and 0.5% Example | 5% w/w benzoyl peroxide and 0.5% w/w |
| rhamnolipid mixture purified by a solvent | Example rhamnolipid mixture (solvent |
| extraction process (25% active) | process) in 20% w/w Tween in dPBS |

The skin analog substrate (Labskin) was prepared as described in Example 4. The C. acnes microorganism was cultured as described in Example 4, and each Labskin unit was inoculated as described in Example 4. The Labskin units were incubated at 37° C. for 24 hours, and after 24 hours, were treated with 11 microliters of control (dPBS), vehicle, or test item. All Labskin units were then incubated at 37° C. for 24 hours, after which biopsy samples of 8 mm in diameter were aseptically removed from the center of each Labskin unit and viable microbial numbers were assayed by recovery on appropriate culture medium.

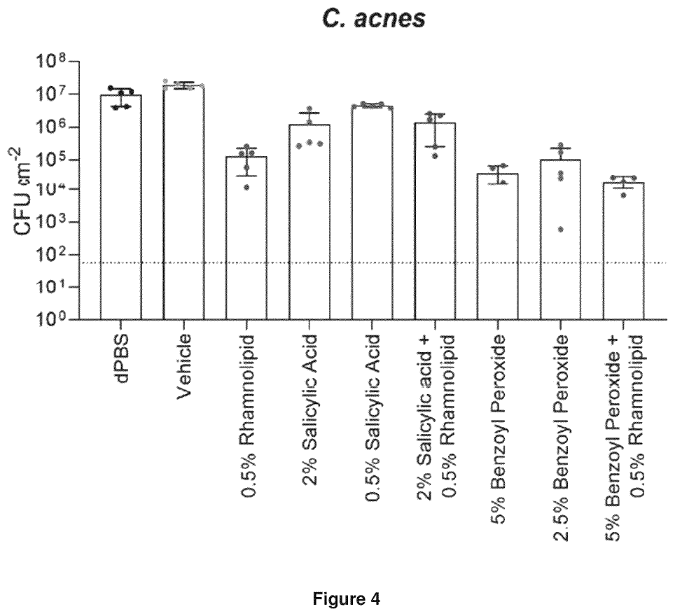

Technical replicate counts obtained from selective solid media were averaged and used to calculate the colony forming units per square cm (CFU cm⁻²). The enumeration of C. acnes for the control, vehicle, and test samples is shown graphically in FIG. 4. The graphed results show that, compared with the dPBS control, there is a slight increase in the average amount of C. acnes shown in the vehicle sample, and a slight decrease in the average amount of C. acnes shown in the 0.5% w/w salicylic acid test sample. All other test samples showed either a noticeable or a significant reduction in the average amount of C. acnes.

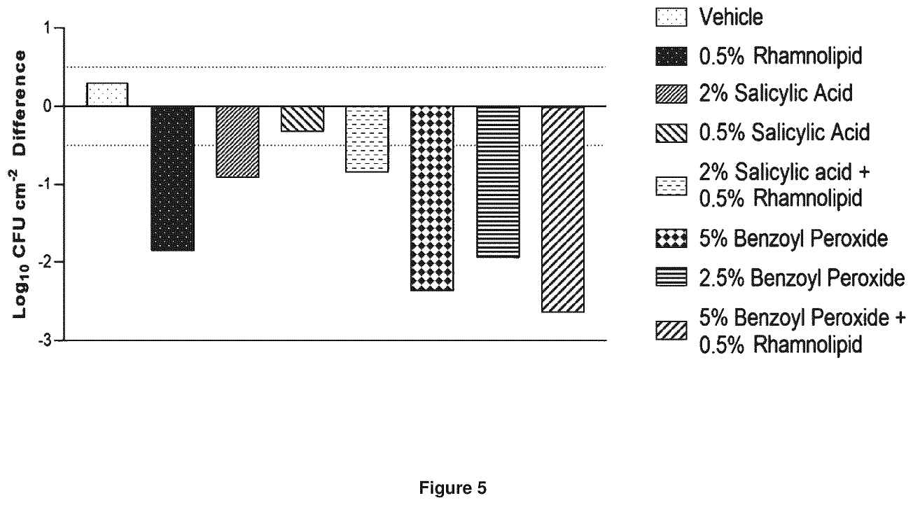

The Log₁₀ difference between the dPBS control and the vehicle and test samples was calculated as described in Example 4, and the Logio difference results are shown graphically in FIG. 5. The graphed results in FIG. 5 show a minor increase in C. acnes recovered from the vehicle sample and a moderate decrease in C. acnes recovered from the 0.5% w/w salicylic acid sample. The samples with 2% w/w salicylic acid and 2% w/w salicylic acid plus 0.5% w/w rhamnolipid showed a noticeable decrease in the recovered population of C. acnes, while the rhamnolipid and benzoyl peroxide samples alone and in combination showed a significant decrease in the recovered population of C. acnes. The results in FIG. 5 also show that the sample containing a concentration of 0.5% w/w rhamnolipids of the present technology was more effective (greater reduction in C. acnes population) than the salicylic acid-containing test samples, and had comparable efficacy to the 2.5% w/w benzoyl peroxide test sample, even though the rhamnolipid concentration was 5× lower than that of the 2.5% w/w benzoyl peroxide sample.

### Example 6: Time-Dependency Comparative Testing

A study was conducted to compare the time-dependent efficacy of a rhamnolipid mixture of the present technology (Example 6 Composition), a commercially available rhamnolipid product (Comparative Composition), and salicylic acid against Cutibacterium acnes (ATCC #6919). The rhamnolipid mixture in the Example 6 Composition was processed using a non-solvent purification process, and had a mono-rhamnolipid to di-rhamnolipid weight ratio in the range of 48:52 to 45:55. The Comparative Composition comprised a mixture of rhamnolipids having a mono-rhamnolipid: di-rhamnolipid ratio of about 4:96 Test samples were prepared by diluting the rhamnolipids and the salicylic acid in sterile water. The concentrations of rhamnolipid actives in the rhamnolipids test samples were 0.003 wt % active, 0.01 wt % active, and 0.03 wt % active. The concentrations of salicylic acid actives in the test samples were 0.03 wt %, 0.1 wt %, and 0.3 wt %. Prior to initiation of the study, samples were sufficiently pre-reduced to remove oxygen by using an indirect atmospheric sequestering of oxygen using hydrogen reaction over palladium catalyst. Test samples were inoculated to a minimum of 1.0E+06 CFU/mL of the C. acnes culture and incubated at 37.0±2.0° C., where aliquots were removed at 15 minutes and 60 minutes. The sample aliquots were neutralized by dilution in an oxygen-reducing buffer containing the flow cytometry dyes, SYTO 9 and Propidium Iodide. Samples were analyzed using flow cytometry where cells were characterized by morphology and viability in comparison to a deionized water control. The results are shown in Table 6.

TABLE 6

| | | | 15 min Exposure | | | 60 min Exposure | | |
|---|---|---|---|---|---|---|---|---|
| | Tested | | Log₁₀ cells/mL | | Percent | Log₁₀ cells/mL | | Percent |
| Samples | pH | Conc. | Recovery | Reduction | Reduction | Recovery | Reduction | Reduction |
| Water Control | 6.7 | N/A | 8.00 | N/A | N/A | 7.30 | N/A | N/A |
| Example 6 | 7.2 | 0.003% | 5.01 | 2.94 | 99.8% | <5.00 | >2.30 | >99.5% |
| Composition | | 0.01% | 5.13 | 2.82 | 99.8% | <5.00 | >2.30 | >99.5% |
| | | 0.03% | <5.00 | >2.95 | >99.8% | <5.00 | >2.30 | >99.5% |
| Comparative | 7.3 | 0.003% | 7.49 | 0.46 | 65.3% | <5.00 | >2.30 | >99.5% |
| Composition | | 0.01% | 5.07 | 2.88 | 99.8% | <5.00 | >2.30 | >99.5% |
| | | 0.03% | <5.00 | >2.95 | >99.8% | <5.00 | >2.30 | >99.5% |
| Salicylic Acid | 2.7 | 0.03% | 7.94 | 0.10 | 20.6% | 6.91 | 0.35 | 55.2% |
| | | 0.10% | 7.89 | 0.10 | 20.6% | 6.12 | 1.14 | 92.8% |
| | | 0.30% | 7.37 | 0.57 | 73.2% | <5.00 | >2.30 | >99.5% |

¹Comparative test sample was pH adjusted to be comparable to the Example 7 Composition test sample.
²Greater than equals maximum log reduction detection limit.

The results in Table 6 show that the Example 6 Composition has better efficacy than the Comparative Composition at an equal concentration of percent active at 0.003% after 15 minutes. The results also show that the Example 6 Composition at a concentration of 0.003% has better efficacy than the 0.30% concentration of salicylic acid after 15 minutes.

### Example 7: Formulated Product Efficacy

A study was conducted to compare the time-dependent efficacy of a formulated product containing the rhamnolipid mixture of the present technology (Example 7 Composition), and commercially available acne treatment products: Neutrogena Stubborn Acne AM Treatment (benzoyl peroxide acne treatment) and Clean & Clear Advantage Acne Spot Treatment (salicylic acid acne treatment) against Cutibacterium acnes (ATCC #6919). The Example 7 Composition comprised an acne base composition and different concentrations of a rhamnolipid mixture of the present technology. The acne base composition comprised propylene glycol in an amount in the range of 57.67% to 60% by weight, xanthan gum in an amount in the range of 1.92% to 2% by weight, a mixture of benzyl alcohol/benzoic acid/dehydroacetic acid in an amount in the range of 0.72% 0.75% by weight, and water to total 100% by weight of the composition. The rhamnolipid mixture was processed using a non-solvent purification process, and had a mono-rhamnolipid to di-rhamnolipid weight ratio in the range of 48:52 to 45:55.

The formulated product and commercial product samples were tested without dilution at product actives concentrations of 0.0% (base composition only), 0.1% and 1.0% rhamnolipids for the Example 7 Composition, 2.5% for the benzoyl peroxide treatment, and 2.0% for the salicylic acid treatment. The formulated product and commercial product samples were also diluted in 75% deionized water to actives concentrations of 0.025% and 0.25% for the Example 7 Composition, 0.625% for the benzoyl peroxide treatment, and 0.50% for the salicylic acid treatment. The product samples were inoculated to a minimum of 1.0E+06 CFU/mL of the C. acnes culture and incubated at 37.0±2.0° C. for 15 minutes. Test sample aliquots were neutralized by dilution in an oxygen-reducing buffer containing the flow cytometry dyes, SYTO 9 and Propidium Iodide. Samples were analyzed using flow cytometry where cells were characterized by morphology and viability in comparison to a deionized water control. The results for the undiluted upfront test samples are shown in Table 7, and the results for the diluted test samples are shown in Table 8.

TABLE 7

| | Samples | | 15 min Exposure | | |
|---|---|---|---|---|---|
| | | Concentration | Log₁₀ cells/mL | | Percent |
| Samples | pH | of Active | Recovery | Reduction | Reduction |
| Water Control | 9.6 | N/A | 7.7 | N/A | N/A |
| Example 7 | 5.5 | 0.00% | 5.8 | 1.9 | 98.84% |
| Composition | | 0.10% | <3.4 | >4.0 | >99.99% |
| | | 1.00% | <3.4 | >4.0 | >99.99% |
| National | 5.0 | 2.50% | <3.4 | >4.0 | >99.99% |
| Benchmark | | | | | |
| (Benzoyl | | | | | |
| Peroxide) | | | | | |
| National | 3.8 | 2.00% | <3.4 | >4.0 | >99.99% |
| Benchmark | | | | | |
| (Salicylic | | | | | |
| Acid) | | | | | |

¹Samples tested were diluted insignificantly (<0.1%) with C. acnes.
²Greater than equals maximum log reduction detection limit.

The results in Table 7 show that the Example 7 formulated product composition at both the 0.10% and 1% active weight concentrations of rhamnolipids performed comparable to the national benchmarks containing 2.5% benzoyl peroxide at pH 5.0 and 2.0% salicylic acid at pH 3.8.

TABLE 8

| | Diluted Samples | | 15 min Exposure | | |
|---|---|---|---|---|---|
| | | Concentration | Log₁₀ cells/mL | | Percent |
| Samples | pH | of Active | Recovery | Reduction | Reduction |
| Water Control | 9.6 | N/A | 7.5 | N/A | N/A |
| Example 7 | 5.5 | 0.00% | 7.1 | 0.4 | 59.9% |
| Composition- | | 0.03% | 4.4 | 3.1 | 99.9% |
| Diluted | | 0.25% | <4.0 | >3.5 | >99.9% |
| National | 5.0 | 0.63% | <4.0 | >3.5 | >99.9% |
| Benchmark | | | | | |
| (Benzoyl | | | | | |
| Peroxide) | | | | | |
| National | 3.8 | 0.50% | <4.0 | >3.5 | >99.9% |
| Benchmark | | | | | |
| (Salicylic | | | | | |
| Acid) | | | | | |

¹Samples were diluted to receive a total volume of 75% sterile deionized water.
²Greater than equals maximum log reduction detection limit.

The results in Table 8 show that the Example 7 Composition, diluted to a rhamnolipid concentration of 0.25%, performs as well as national benchmarks containing 0.625% benzoyl peroxide at pH 5.0 and 0.5% salicylic acid at pH 3.8.

The embodiments and examples described here are illustrative, and do not limit the presently described technology in any way. The scope of the present technology described in this specification is the full scope defined or implied by the claims. Additionally, any references noted in the detailed description section of the instant application are hereby incorporated by reference in their entireties, unless otherwise noted.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further, the examples are provided to not be exhaustive but illustrative of several embodiments that fall within the scope of the claims.

What is claimed is:

1. An acne treatment composition comprising:
(a) a mixture of purified rhamnolipid salts in an amount of about 0.003% to about 6% by weight, based on the total weight of the composition, wherein the mixture of purified rhamnolipid salts comprises mono-rhamnolipids and di-rhamnolipids in a weight ratio of 10:90 to 47:53 mono-rhamnolipids: di-rhamnolipids, an amount of Rha-C10-C10 mono-rhamnolipid salt of about 5% to about 38% by weight, based on the total weight of the mixture of rhamnolipid salts, and an amount of RhaRha-C10-C10 di-rhamnolipid salt of about 34% to about 80% by weight, based on the total weight of the mixture of rhamnolipid salts; and
(b) at least one dermatologically acceptable carrier, and optionally one or more additives, in an amount to total 100% by weight of the composition.

2. The acne treatment composition of claim 1, wherein the weight ratio of mono-rhamnolipids to di-rhamnolipids is 40:60 to 47:53.

3. The acne treatment composition of claim 1, wherein the amount of Rha-C10-C10 mono-rhamnolipid salt is about 29% to about 37.5% by weight based on the total weight of the mixture of rhamnolipid salts.

4. The acne treatment composition of claim 1, wherein the amount of RhaRha-C10-C10 di-rhamnolipid salt is about 36% to about 45% by weight based on the total weight of the mixture of rhamnolipids.

5. The acne treatment composition of claim 1, wherein the composition comprises an amount of RhaRha-C10-C12 di-rhamnolipid salt of about 5% to about 15% by weight based on the total weight of the mixture of rhamnolipids.

6. The acne treatment composition of claim 1, wherein the composition comprises an amount of RhaRha-C10-C12:1 di-rhamnolipid salt of about 0.2% to about 5% by weight based on the total weight of the mixture of rhamnolipids.

7. The acne treatment composition of claim 1, wherein the composition comprises an amount of RhaRha-C8-C10 di-rhamnolipid salt of about 0.2% to about 5% by weight based on the total weight of the mixture of rhamnolipids.

8. The acne treatment composition of claim 1, wherein the composition comprises an amount of Rha-C10-C12 mono-rhamnolipid salt of about 0.2% to about 6% by weight based on the total weight of the mixture of rhamnolipids.

9. The acne treatment composition of claim 1, wherein the composition comprises an amount of Rha-C8-C10 mono-rhamnolipid salt of about 0.2% to about 5% by weight based on the total weight of the mixture of rhamnolipids.

10. The acne treatment composition of claim 1, wherein the one or more additives comprise one or more of surfactants, pH adjustment agents, skin conditioners, antioxidants, preservatives, fragrances, pigments, and dyes.

11. The acne treatment composition of claim 1, wherein the dermatologically acceptable carrier comprises water.

12. The acne treatment composition of claim 1, wherein the dermatologically acceptable carrier comprises alcohols, glycols, solvents, emulsifiers, solubilizers, vegetable gums, starches, celluloses, waxes, silicone, silica, and clays.

13. The acne treatment composition of claim 1, wherein the composition has a pH of 4.0 to 8.5.

14. The acne treatment composition of claim 1, wherein the composition is an aqueous solution, suspension, cream, lotion, gel, paste, spray, cream, foam, emollient, pad, or wipe.

15. The acne treatment composition of claim 1, wherein the composition comprises a co-active component in combination with the mixture of rhamnolipid salts; wherein the co-active component is salicylic acid, benzoyl peroxide, resorcinol, resorcinol monacetate, or sulfur, or any combination thereof.

16. The acne treatment composition of claim 1, wherein the composition exhibits a percent reduction in C. acnes Colony Forming Units (CFU) per mL of about 70% to about 99.999% at a rhamnolipid concentration of 0.25% by weight of the composition at a contact time of 15 minutes.

17. The acne treatment composition of claim 1, wherein the composition exhibits a Log₁₀ Reduction in C. acnes Colony Forming Units (CFU) per cm⁻² of about 0.52 to about 5.0 at a rhamnolipid concentration of 0.25% by weight of the composition at a contact time of 15 minutes.

18. The acne treatment composition of claim 1, wherein the mixture of purified rhamnolipid salts exhibits a Minimum Inhibitory Concentration (MIC) for C. acnes of about 21 ppm to about 98 ppm.

19. The acne treatment composition of claim 1, wherein the mixture of purified rhamnolipid salts exhibits a Minimum Bacteriocidal Concentration (MBC) for C. acnes of about 61 ppm to about 296 ppm.

20. A method of controlling, treating or preventing acne in a subject afflicted with or susceptible to acne comprising applying to the skin of the subject an effective amount of a composition comprising a mixture of purified rhamnolipid salts, thereby controlling, treating or preventing acne in the subject, wherein the mixture of purified rhamnolipid salts is present in the composition in an amount of 0.003% to 6.0% by weight, based on the total weight of the composition and comprises mono-rhamnolipid salts and di-rhamnolipid salts in a weight ratio of 10:90 to 47:53 mono-rhamnolipids: di-rhamnolipids, an amount of Rha-C10-C10 mono-rhamnolipid salt of about 5% to about 38% by weight, based on the total weight of the mixture of rhamnolipids, and an amount of RhaRha-C10-C10 di-rhamnolipid salt of about 34% to about 80% by weight, based on the total weight of the mixture of rhamnolipid salts.
